(19) United States
(12) Patent Application Publication
(10) Pub. No.: US 2009/0054377 A1
(43) Pub. Date: Feb. 26, 2009

(54) COMPOSITIONS AND METHODS FOR SMOKING CESSATION

(75) Inventor: Avner Shenfeld, Rehovot (IL)

Correspondence Address: THE NATH LAW GROUP, 112 South West Street, Alexandria, VA, 22314, US

(73) Assignee: MODUS BIOLOGICAL MEMBRANES LTD., Rehovot (IL)

(21) Appl. No.: 11/658,665

(22) PCT Filed: Jul. 25, 2005
(86) PCT No.: PCT/IL2005/000789

§ 371 Date: Oct. 29, 2008

Related U.S. Application Data
(60) Provisional application No. 60/591,261, filed on Jul. 27, 2004.
(60) Provisional application No. 60/659,095, filed on Mar. 8, 2005.

(51) Int. Cl. A61K 31/661; A61P 25/34

(52) U.S. Cl. 514/114; 514/121

(57) ABSTRACT

The invention discloses a method and a composition for smoking cessation or for promoting smoking cessation in individual smokers. The compositions described contain phosphatidic acid as the active ingredient.

### FIELD OF THE INVENTION

The present invention relates to compositions and methods for smoking cessation.

### BACKGROUND OF THE INVENTION

Smoking addictions as well as addictions to drugs of abuse are known to involve cognition enhancement, physiological conditioning, stress adaptation and relief from withdrawal. The combination of all these factors makes smoking cessation extremely difficult, even for well motivated individuals who are aware of the extensive damage to health caused by cigarette and other tobacco smoke inhaling devices.

A recent meta-analysis of 17 published smoking cessation trials has determined that only 27% out of 5098 smokers, treated by various regimens, remained abstinent at the end of treatment, as compared to 13% in the placebo groups. Even intensive inpatient treatment resulted in only 29% biochemically verified abstinence after one year of follow-up.

One of the main obstacles in achieving successful weaning from smoking is the rapid development of severe withdrawal symptoms, similar to those experienced in all chemical addictions, upon withdrawal of the abused substances. The symptoms are both physiological and psychological. The main symptoms include dizziness, chest tightness, constipation, cough, mouth sores, anxiety, irritability, sleep disturbances, confusion, eating disorders and a persistent craving for a cigarette. Women smokers especially dread the almost irresistible increase in appetite, often resulting in a rapid weight increase. Changes in pulse and blood pressure have been reported in smokers, as well as chronic bone-marrow stimulation, resulting in increases in hemoglobin levels and leukocytosis (Bain B J et al; Acute changes in haematological parameters on cessation of smoking. J R Soc Med, 1992, 85:80-82). These changes are gradually normalized following smoking cessation.

Various techniques have been applied for assisting the smoker to overcome the withdrawal syndrome occurring in the early stage of smoking cessation. They range from chemical substitutes, such as widely used nicotine preparations, clonidine, to psychological conditioning and more recently anti-depressive pharmacotherapy. Tobacco abstinence may be monitored by biochemical parameters, such as blood cotinine levels and the measurement of exhaled carbon monoxide, self-reported cigarette-smoking per day (CPD) and the administration of questionnaires, such as the Fagerstrom Tolerance Questionnaire (FTQ), or the improved Fagerstrom Tolerance Nicotine Dependence (FTND) questionnaire.

Pharmacological interventions are associated with dose-dependent side-effects. Those are mainly observed with nicotine transdermal patches, from which absorption is uncontrolled and may lead to palpitations, tremor, and restlessness, amongst others. Paradoxically, these symptoms are similar to those of the withdrawal syndrome itself and may be particularly hazardous in patients with a compromised cardiovascular system, who actually are the ones most in need of smoking cessation.

As already stated, the overall results of smoking cessation treatments remain rather discouraging. It seems that the availability of a non-toxic agent, easy administration routes and the capability to suppress the withdrawal symptoms, may significantly improve the outcome of smoking cessation.

One hypothesis, attempting to explain the pathophysiology of the abused substance withdrawal syndrome, as tested in mice, claims that it is caused by a loss of fluidity of central nervous system (CNS) cell-membranes, upon lack of incorporation of the addictive chemical into the membrane. The loss of fluidity is associated with a decrease in the cholesterol content of the membrane (Heron S, Shinitzky M, Samuel D; Alleviation of drug withdrawal symptoms by treatment with a potent mixture of natural lipids. Eur. J. Pharmacol., 1982, 83:253-6). Craving for the addictive substance is relieved when it is supplied to the cell, reversing the leak of cholesterol. It has been suggested that similar changes occur in the post-menopausal syndrome and in the process of aging, due to hormonal deprivation. Indeed some of the symptoms in these conditions, such as confusion, sleep disturbances, irritability and loss of memory, resemble those of the withdrawal syndrome (Shinitzky et al, unpublished).

U.S. Pat. No. 6,051,564 to Shenfeld and Shinitzky discloses phospholipid compositions containing phosphatidic acid and their use in a method for the treatment of withdrawal symptoms including such associates with rehabilitation from smoking. These compositions were shown to assist individuals in overcoming several of the withdrawal symptoms and eventually in reducing the number of cigarettes smoked per day at the end of a three-week study.

### SUMMARY OF THE INVENTION

It has now been found that a phospholipid composition comprising at least about 7% phosphatidic acid (PA) may be useful in promoting cessation of smoking. In particular the compositions do not comprise more than 75% PA.

Within the context of the present invention, the term “smoking cessation” refers to the FDA-defined smoking cessation, which refers to complete abstinence from smoking cigarettes or other forms of tobacco for a period of at least 28 consecutive days prior to the end of the clinical study. Compliance is typically verified by measuring exhaled CO levels. Within the scope of the present invention, the term also includes nearly complete abstinence from smoking during the period indicated.

The term “smoking cessation treatment” refers to a treatment within the scope of the present invention that would ensue abstinence from smoking. Such treatment may or may not be associated with any one or more withdrawal symptoms, or with any one or more other physiological or psychological symptom, as detailed below.

Thus, in one aspect of the present invention, a composition to be used by individual smokers for promoting cessation of smoking is provided, said composition comprises at least about 7% phosphatidic acid (PA). Typically, the composition comprises between at least about 7% to at least about 75%.

In one embodiment of the present invention, the PA composition comprises between at least about 10% to at least about 50% PA. Preferably, the composition comprises between at least about 15% to at least about 30% PA. More preferably, the composition comprises between at least about 15% to at least about 25% PA. Most preferably, the compositions comprise at least about 16%, 17%, 18%, 19%, 20%, 21% or 22% PA.

The PA composition may be administered simultaneously with a known smoking cessation agent such as Zyban, i.e., as one medicament or as two individual medicaments taken at the same time or one within a short time after the other, or they may be taken sequentially before or after a known said smoking cessation agent, in order to achieve a greater effect and/or longer cessation period. Due to the possible side effects of such smoking cessation agents, their dosage may be reduced to minimum when administered simultaneously with or sequentially to the composition of the present invention.

The present invention also provides PA compositions, as disclosed hereinbefore, which further comprise at least one chemical selected from antioxidants such as Vitamins and beta-carotene and phospholipids from an animal or vegetable source, such as phospshatidylserine (PS). The at least one chemical may be presented in its salt form or in any other form so as to minimize disruptive interaction with the PA present in the composition. The at least one chemical may also be administered separately from the PA composition, in a sequential manner or simultaneously thereto.

The term “antioxidant” refers to a chemical that prevents the oxidation of other chemicals and prevents free radicals from damaging essential molecules. Such antioxidant chemical may be vitamins such as: Retinol (Vitamin A or beta-carotene); Ascorbic acid (Vitamin C); and Vitamin E. Another antioxidant is Selenium. The use of the appropriate antioxidant to be used in combination with the composition of the present invention may be chosen in view of the clinical condition of the subject and other parameters known to a medical practitioner.

The PA and the at least one chemical, e.g. vitamin or PS, may be present in the composition in various ratios. In one embodiment, the composition of the present invention comprises PA and PS in a ratio of 1:1. In another embodiment, the composition comprises PA and PS in a ratio of 1:0.5, respectively. In yet another embodiment, the composition may comprise PA and PS in a PA:PS ratio of 1:1.5 or 1:2, respectively. It should be understood that other PA:PS ratios are also possible and fall within the scope of the present invention.

The compositions of the present invention typically comprise 0.5 to 4 grams of said PA mixture. Preferably, the composition comprises between 0.5 and 2 grams; most preferably between 0.5 to 1 grams of the PA mixture.

The compositions of the present invention are effective even in cases where prior smoking cessation attempts had not been accompanied by any one side effect. The compositions may also be used with individuals who at the present attempt for smoking cessation are absent of any such side effects. Thus, the compositions of the present invention may be utilized for promoting smoking cessation which may or may not be accompanied or associated with any one substantial side effect such as nausea, sweating, shaking, substance craving, hot flushes, insomnia, dry mouth, dizziness, anxiety, constipation and the like.

In one preferred embodiment, the smoking cessation is not associated with any one or more physiological or psychological side effect.

The composition may be administered orally at dosages ranging from 0.5-4.0 g/day. A preferred dosage is between 0.5 g/day to 2.0 g/day of the composition containing PA. The most preferred dosage is 0.5 g/day to 1.0 g/day. The suitable dosage range, however, may be determined depending, inter alia, on the severity of smoking disorders, frequency of treatment and route of administration. Preferably, the composition is in a unit dosage form. For example, the preparations may be in a pack form accompanied by printed instructions for use in a smoking cessation treatment.

The composition may further comprise a diluent, binder, filler, disintegrate, flavoring agent, coloring agent, lubricant or preservative in a conventional manner, such additional components are known to a person skilled in the art. In one preferred embodiment, the diluent is a mineral oil.

By another aspect of the invention, there is provided a method for smoking cessation treatment which comprises administering to an individual smoker an effective amount of the phosphatidic acid composition of the present invention.

By another aspect, there is provided a method for abstinence from smoking cigarettes or other forms of tobacco for a period of at least 28 consecutive days, said method comprising administering to an individual smoker an effective amount of a phosphatidic acid composition as disclosed hereinbefore.

By yet another aspect of the invention, there is provided a method for promoting smoking cessation which comprises administering to an individual smoker an effective amount of the phosphatidic acid composition of the present invention. The term “promoting smoking cessation” refers to a reduction in the number of cigarettes smoked by the individual treated with the composition of the present invention.

In a further aspect, the present invention provides for a kit for smoking cessation which comprises an administrable form of the composition comprising PA and instructions for use.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 depicts absolute quit rate at the end of the treatment.

FIG. 2 depicts development of the placebo effect versus PA composition effect after two months of treatment.

### DETAILED DESCRIPTION OF THE INVENTION

The phosphatidic acid (PA) content of the compositions of the present invention is a product of partial hydrolysis of lecithin phospholipids using phospholipase-D from peanuts. It is a complex mixture of Hexane-soluble substances which consists of phosphatidic acid, other phospholipids such as phophatidylcholine, phosphatidylnositol, phosphatidylethanolamine, phosphatidylglycerol and lysophospholipids and triglycerides. In the enriched mixture, herein referred to as the “primary PA mixture” the total PA is between 40% and 50%. The concentration given in % units means to denote the number of weight units of the ingredient per 100 weight units of the entire composition (w/w).

The primary PA mixture is preferably obtained from natural phospholipids preparation by enzymatic treatment using synthetic or natural source comprising the enzyme phospholipase-D. One such preparation is disclosed in U.S. Pat. No. 6,051,564 to the same authors of the present invention.

Typical examples of natural phospholipids preparations useful for the preparation of the PA mixture are, without being limited thereto, Soya lecithin, and egg yolk. Typically, the phospholipase-D source is, without being limited thereto, peanuts, cabbage, bacteria or fractions derived therefrom.

The compositions of the present invention are typically prepared by admixing the primary PA mixture with at least one suitable solubilizing agent that is capable of dissolving the highly viscous or at times pasty primary PA mixture. The composition may also comprise other ingredients such as, and without being limited thereto, vitamins, antioxidants, preservatives, stabilizers and the like. The overall concentration of the PA in the compositions of the present application is typically between at least about 7% and at least about 75%. The term “at least about” as used with reference to the concentration of PA in the compositions of the present invention, for example in the expression “at least about 7%”, is meant to mean 7% PA+/−2%, namely compositions comprising 5% may also fall within the scope of the present invention.

The composition of the present invention may be administered in a form of any administrable formulation which may be found suitable. Such “administrable forms” may be, without being limited thereto, such formulations that may be administrated orally, topically, intraperitoneally, intraarticularly, intracranially, intradermally, intramuscularly, intraocularly, intrathecally, intravenously, or subcutaneously, as further detailed hereinbelow. Other methods known to a person skilled in the art are also applicable. In most cases, the composition may be prepared for oral use, for example in oil.

Depending on the formulation used, a suitable pharmaceutically acceptable carrier, excipient or diluent may be used. The pharmaceutically acceptable carriers described herein, for example, vehicles, adjuvants, excipients, or diluents, are well-known to those skilled in the art and are readily available to the public. It is preferred that the pharmaceutically acceptable carrier be one which is chemically inert to the PA and one which has no detrimental side effects or toxicity under the conditions of use.

The choice of a carrier will be determined in part by the particular constitution of the PA mixture, as well as by the particular method used to administer the composition. Accordingly, there is a wide variety of suitable formulations of the pharmaceutical composition of the present invention.

Formulations suitable for oral administration can consist of (a) liquid solutions, such as an effective amount of the compound dissolved in the solubilizing agents or diluents, such as water, saline, oils or orange juice; (b) capsules, sachets, tablets, lozenges, and troches, each containing a predetermined amount of the active composition, as solids or granules; (c) powders; (d) suspensions in an appropriate liquid; and (e) suitable emulsions.

The solubilizing agents used for oral administration are typically selected from the group consisting of mineral or vegetable oils. Preferably, the solubilizing agents are vegetable oils selected from canola oil, corn oil, sunflower seed oil, olive oil, jojoba oil, sesame oil, peanut oil, chili oil, hazelnut oil, walnut oil, and the like. The preferred oils are canola and peanut oils. The most preferred oil is canola oil.

Liquid formulations may include diluents, such as oils as described hereinbefore, water and alcohols such as ethanol, benzyl alcohol, and the polyethylene alcohols, either with or without the addition of a pharmaceutically acceptable surfactant, suspending agent, or emulsifying agent.

Capsule forms can be of the ordinary hard- or soft-shelled gelatin type containing, for example, surfactants, lubricants, and inert fillers, such as lactose, sucrose, calcium phosphate, and cornstarch.

Tablet forms may include for example one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers.

Lozenge forms can comprise the PA mixture in a flavor, usually sucrose and acacia or tragacanth, as well as pastilles comprising the PA mixture in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The composition may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compositions of the present invention may be made into injectable formulations. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds, pages 238-250 (1982), and ASHP Handbook on Injectable Drugs, Toissel, 4^{th} ed., pages 622-630 (1986).

Additionally, the compositions of the present invention may be made into suppositories by mixing with a variety of bases, such as emulsifying bases or water-soluble bases.

The compositions of the present invention may be administered in combination with another smoking cessation remedy such as those known in the market as over-the counter (OTC) remedies or as prescription drugs. Examples of OTC remedies are paracetamol, loratidine. Examples of prescribed remedies are cephalosporin antibiotics, atenolol, simvastatin.

The compositions may also be given in combination with physiological treatment in order to strengthen the subject treatment.

In a further aspect, the present invention provides for a kit for smoking cessation which comprises an administrable form of the composition comprising PA and instructions for use.

### EXAMPLE 1

### PA Composition

Capsules made of gelatin (Type LB, Gel Formula 3A), size 22, oblong, brown colored (151A) were used. Each capsule contained: PA mixture, oil and vitamin E to a total weight of 1000 mg. The total PA in such a composition was between 15-25% w/w.

Formulation 1 contained 7% PA: 400 mg canola oil, about 50-80 mg phosphatidic acid, Vitamin E and about 440 mg of other phospholipids.

Formulation 2 contained 16% PA: 400 mg canola oil, about 160 mg phosphatidic acid, 3.5 mg Vitamin E, and about 436 mg other phospholipids.

Formulation 3 contained 18% PA: 400-500 mg canola oil or peanut oil, about 180 mg phosphatidic acid, 3.5 mg Vitamin E, and about 300-407 mg other phospholipids.

Formulation 4 contained 22% PA: 400-500 mg canola oil, about 200-220 mg phosphatidic acid, 3.5 mg Vitamin E, and about 300-400 mg other phospholipids.

Formulation 5 contained equal weight amounts of PA and PS (ratio of 1:1): such a formulation is prepared by mixing PA and PS with, for example, canola oil as a carrier. Other ingredients such as Vitamin E may also be added.

The formulations generally contained 6-8% phosphatidylcholine, 9-11% phosphatidylethanolamine, 8-10% phosphatidylinositole, about 20% other phospholipids and about 40% triglycerides. Formulation analysis of the compositions gave the additional following data:
1. Free acid value—not more than 7.
2. Peroxide value—not more than 10 (based on 5 gram samples).
3. Heavy metals—not more than 20 ppm.
4. Residual solvents—not more than 290 ppm hexane.
5. Microbial contamination—negative to E. Coli and Salmonellae

### EXAMPLE 2

### In Vivo Studies

Study 1: Forty one smokers of more than 10 cigarettes per day (CPD), healthy men or (non-pregnant) women, who admitted to smoking for at least 1 year and who were unable to stop smoking, completed a double-blind, placebo controlled study (placebo containing triglyceride only), in which a single dose, given twice daily of a composition containing varying amounts of 15-25% phosphatidic acid (PA) in triglyceride, was administered orally in a liquid preparation over a period of 3 weeks, starting two days prior to smoking cessation.

In one test group, there were 19 males and 22 females, 20 in the active treatment group and 21 in the placebo group. Nicotine dependency and withdrawal symptoms were assessed by questionnaires adapted from the Fagerstrom Tolerance Questionnaire (FTQ). A final score ranging from 1-5 was applied for statistical evaluation. A reduction in the craving for food (p<0.05) after 1 week and a significant decrease in the craving for cigarettes (p<0.05) after 3 weeks, were observed in the PA-treated group, as compared to the placebo-treated group. A total of 10/18 individuals (66%, or 10/20 intent to treat—50%) were completely abstinent (CPD=0) after 3 weeks of treatment with the PA composition, as compared to 3/19 in the placebo group (15.5%, or 3/21 intent to treat—14.2%).

Study 2: On the basis of the results of the previous studies, a Phase IIB trial was completed in Israel, according to the FDA regulatory requirements. The study was monitored by an accredited CRO (Quintiles Ltd). The protocol was developed according to the template of the decisive Zyban clinical trial (Jorenby D E et al, A controlled trial of sustained-release bupropion, a nicotine patch or both for smoking cessation. New Engl. J. Med. 1999, 340:685-91) and was approved by the FDA consultant of Quintiles.

The study was randomly and equally divided between two different sites in Israel. Two teams of physicians and nurses were specially trained for the task. 493 smokers of ≧10 cigarettes/day (CPD) during ≧1 year, participated in a double-blind, randomized, 5 arm, dose finding, smoking-cessation trial, in which gelatin capsules of the formulations above were administered during 63 days, followed by 28 days of follow-up without treatment. The daily doses were 4.0 g, 2.0 g, 1.0 g, 0.5 g of the PA composition and placebo. A complete hemogram, standard blood biochemistry, including liver and renal functions, were taken upon enrollment prior to treatment initiation and on days 56 and 84 of the study. All adverse events were recorded. The primary end-point was a complete smoking abstinence during ≧28 days (FDA definition), or smoking quit rate (SQR). Abstinence was verified by measurements of exhaled CO.

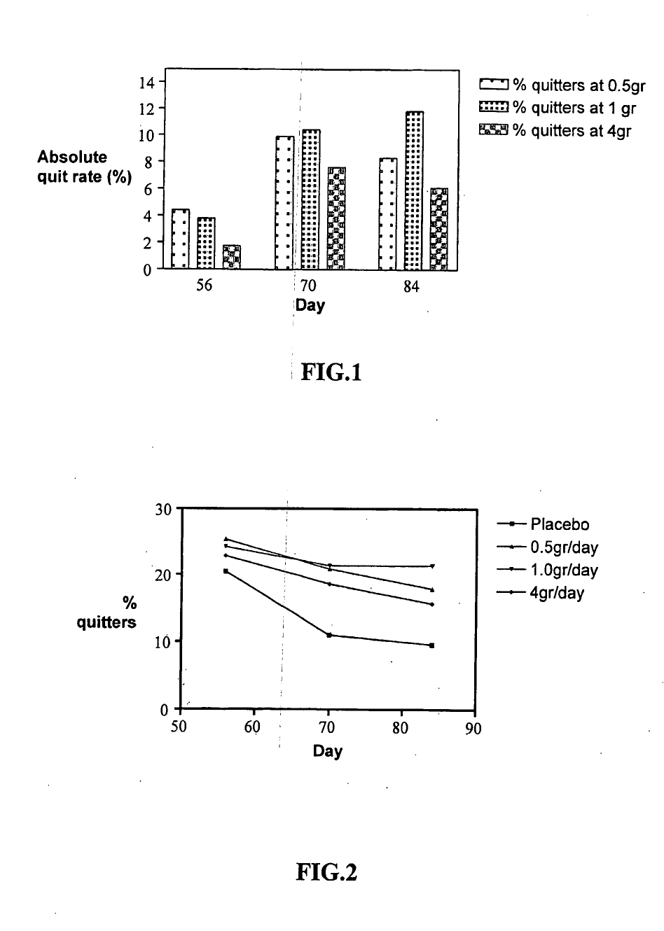

The results of the SQR percentages are summarized in Table 1 and visualized in the FIG. 1.

TABLE 1
Smoking Quit Rate results.

| Treatment Group- | | | |
|---|---|---|---|
| g/day | Day 56 | Day 70 | Day 84 |
| 0 (placebo) | 20.5% | 11.0% | 9.6% |
| 0.5 | 25.4% | 20.9% | 17.9% |
| 1.0 | 24.3% | 21.4% | 21.4% |
| 4.0 | 22.9% | 18.6% | 15.7% |

FIG. 1 depicts the SQR after deducting the value of placebo from the total SQR for each day. As may be noted from Table 1 and FIG. 1, the results in the group of 2.0 g are not presented, due to an abnormal skewing of the SQR to only 7%. Demographic analysis showed that in spite of meticulous randomization this group contained a higher percentage of elderly men who smoked less than 30/CPD. These features may have affected negatively the compliance in this group. Overall, the best and most significant difference is achieved in the 1.0 g group on Day 84, after one month without treatment, with the waning of the placebo effect. The net SQR (minus placebo) is 11.8% with an Odds Ratio (OR) of 2.57.

These results are within the same range as nasally administered Nicotine Replacement Therapy (NRT) and Zyban, however with a much better safety profile. Zyban's adverse events include insomnia, seizures, and hallucinations and lately has been suspected to be associated with over 50 cases of death. The NRT may mainly cause cardiovascular effects, such as palpitations and elevations in blood pressure.

No adverse-effect of the PA composition of the present invention was noted on the hematological and biochemical profile of the participants. About 5% of the participants exhibited mild side effects which were equally distributed between all the groups, mainly related to the gastrointestinal tract (as anticipated), such as nausea, some loose stools and a transient abdominal discomfort (Table 3).

In FIG. 2 the data represented in Table 1 is shown as a graphic representation. The absolute number of quitters was reduced by half and was nearly constant at the end of the treatment period, providing an indication to the inability of the placebo to maintain prolonged abstinence. In comparison, the absolute number of quitters who were treated with various concentration of PA decreased to only a smaller extent. The absolute number of individuals treated with 1.0 g PA per day remained nearly the same after the treatment was stopped, which is an indication to the ability of the PA composition to maintain prolonged abstinence.

The ability of the composition of the instant invention in prolonging abstinence may be concluded also from the study which compares the absolute quite rate of the PA composition with over-the counter remedies for nicotine-related therapies.

TABLE 2
comparison between the absolute smoking quit rate using PA
compositions and other smoking cessation remedies (Absolute
quit rate = quit rate − quit rate of placebo)

| Product | % Quitters | Absolute quit rate |
|---|---|---|
| Gum | 18.2 | 7.6 |
| Patches | 20.5 | 9.7 |
| Nasal spray | 25.9 | 16.0 |
| Inhaler | 15.2 | 10.2 |
| Bupropion (Zyban) | 22.5 | 13.5 |
| Bupropion (Zyban) | 25 | 11 |
| 300 mg/day | | |
| PA Composition | 21.4 | 11.8 |
| (1 gr/day) | | |

As Table 2 summarizes, the PA composition of the present invention is at least as efficient as known remedies for cessation of smoking such as Zyban. The efficiency is better appreciated when the toxicity and side effects of the composition (Table 3) are compared with those associated with the other remedies known in the market. The major side effect reported by the subject participants was abdominal comfort (13% as compared to 6% by those treated by placebo). Other side effects reported were nausea (3%, placebo 4%) and heartburn (2%, placebo 0%).

TABLE 3
side effects associated with each smoking cessation remedy.

| Adverse Events | Placebo % | Zyban % | NRT % | PA % |
|---|---|---|---|---|
| Insomnia | 18-21 | 31-40 | 28 | 3 |
| Dry Mouth | 4-5 | 1-11 | 4 | 0 |
| Rhinitis | 8 | 12 | 11 | 0 |
| Dizziness | 6-7 | 8-10 | 2 | 0 |
| Anxiety | 6 | 8 | 6 | 0 |
| Constipation | 3 | 8 | 6 | 2 |
| Diarrhea | 4 | 4 | 1 | 2 (soft stool) |
| Dream abnormality | 3 | 5 | 18 | 0 |
| Seizures | 0 | 0.4 | 0 | 0 |

No changes in the hemogram, lymphocyte subpopulations, and routine biochemistry and immunoglobulin levels were noted during the study in either treatment group. A screening of some serum cytokines, which was performed by commercial ELISA kits (enzyme-linked immunosorbent assay) in study 1, revealed an elevation in levels of interferon gamma (IFN-γ) after treatment with the PA composition.

Study 3: On the basis of the above protocol, a composition comprising 13% PA was given to a group of 45 subjects for a period of 84 days. At the end of day 56, a total of 12 subjects dropped out of the group. Out of the remaining 33 participants, 18 (55%) completely stopped smoking and 6 (18%) reduced the number of cigarettes smoked per day from more than 30 to 5/day. The remaining 9 participants continued smoking.

### EXAMPLE 3

### Weight Gain and Smoking Cessation

Weight gain is one of the most feared from side effect associated with smoking cessation. As will be shown hereinbelow, treatment with the compositions of the present invention leads to a much smaller weight gain for subjects who stopped smoking as compared with the placebo treated subjects.

Weight gain was measured for subjects who reached the end of the study (so called “per protocol”) and was calculated as weight at day 70 (the last weighing procedure) minus weight at day 7 (the first weighing procedure). As shown in Table 4, among the “treated” versus “placebo” groups, the subjects who stopped smoking while receiving placebo gained 3.42 kg while the subjects treated with compositions comprising 0.5 grams or 1.0 grams PA gained on average 1.52 kg (P=0.006).

Another surprising observation is the effect of heavy smokers versus light smokers. As may be noticed from Table 4, if “treated” versus “placebo” is compared for both heavy smokers and light smokers, it is evident that the effect of the composition on heavy smokers was greater as compared to light smokers.

This observation is clinically surprising and is of great importance for heavy smokers who traditionally believed, as did the medical professionals, that it is more difficult for heavy smokers to stop smoking in comparison to those who smoke less cigarettes per day.

TABLE 4
A summary of the average weight gains (in Kg)
of subjects per category as analyzed.

| | | Stopped |
|---|---|---|
| Heavy (>30 | Treated | 1.83 |
| cigarettes/day) | Placebo | 4.00 |
| | Total | 2.92 |
| Light (<30 | Treated | 1.20 |
| cigarettes/day) | Placebo | 2.83 |
| | Total | 2.02 |
| Total | Treated | 1.52 |
| | Placebo | 3.42 |
| | Total | 2.47 |

### EXAMPLE 4

### COHb Levels as a Control Measurement of Smoking Cessation

In all smoking cessation studies, there may be a problem of reporting credibility. Subjects may report having stopped smoking when actually they have not. This may happen when a subject intends to please the investigator or when there is a reluctance to admit failure. The opposite case may also occur, especially when a subject is displeased with the study conduct and has therefore a wish to hinder regular progress. In order to minimize such risks, CO checks were performed and recorded at each of a subject's visits to the clinic.

Instruments used for this purpose, measure the concentration of CO in parts per million (ppm) of exhaled air after a subject holds breath for some 20 seconds. The obtained values are converted to percent COHb in the blood assuming balanced conditions are obtained while holding breath. Experience shows that COHb values smaller than 11% are consistent with no smoking, while higher values are consistent with smoking.

The CO measurements were performed at the last visit (6^{th} visit) made on day 84 from the start of the study. All available data for this visit pertains to 354 subjects. In Table 5 below, data showing the numbers of subjects who declared smoking cessation is compared with the number of those who did not and their respective CO measurements:

TABLE 5
A summary of data showing the numbers of subjects
who declared smoking cessation, the number of those
who did not and their respective CO measurements.

| | COHb < 11% | COHb > 11% | Total |
|---|---|---|---|
| Subjects declared having | 50 | 2 | 52 |
| stopped | | | |
| Subjects declared not having | 22 | 280 | 302 |
| stopped | | | |
| Total | 72 | 282 | 354 |

Fisher's Exact Rest was used to check the association between the data shown. The value obtained was P<0.0001 which is very significant. Positive and negative predictive values were calculated to yield: a positive predictive value of 0.9615 with 95% confidence limits of 0.8680 to 0.9953, and a negative predictive value of 0.9272 with 95% confidence limits of 0.8916 to 0.9537. These results indicate that in the herein disclosed study, a very good correlation was found between the self-reporting of smoking cessation or non-cessation and the independent COHb measurements.

1-73. (canceled)

74. A pharmaceutical composition for smoking cessation or promoting smoking cessation comprising at least about 7% (w/w) phosphatidic acid (PA) and a pharmaceutically acceptable diluent, binder, filler, disintegrate, flavoring agent, coloring agent, lubricant or preservative.

75. The composition of claim 74 comprising between about 7% and at least about 75% phosphatidic acid.

76. The composition according to claim 75 comprising between at least about 15% and at least about 50% phosphatidic acid.

77. The composition according to claim 75 comprising between at least about 15% and at least about 30% phosphatidic acid.

78. The composition according to claim 75 comprising between at least about 15% and at least about 25% phosphatidic acid.

79. The composition according to claim 74, wherein said smoking cessation is not associated with any one substantial side effect.

80. The composition according to claim 74, wherein said smoking cessation is associated with at least one side effect.

81. The composition according to claim 79, wherein said side effects are selected from nausea, sweating, shaking, substance craving, hot flushes, insomnia, dry mouth, dizziness, anxiety, and constipation.

82. The composition according to claim 80, wherein said side effects are selected from nausea, sweating, shaking, substance craving, hot flushes, insomnia, dry mouth, dizziness, anxiety, and constipation.

83. The composition according to claim 74 in an administrable form.

84. The composition according to claim 83, wherein said administrable form is selected from the group consisting of oral, topical, intraperitoneal, intraarticular, intracranial, intradermal, intramuscular, intraocular, intrathecal, intravenous, and subcutaneous.

85. The composition according to claim 84 adapted for oral administration.

86. The composition according to claim 74 further comprising at least one vitamin.

87. The composition according to claim 74 further comprising phosphatidylserine (PS).

88. A method for promoting smoking cessation, said method comprising administering to an individual smoker an effective amount of a composition comprising phosphatidic acid.

89. A method for smoking cessation treatment, said method comprises administering to an individual smoker an effective amount of a composition comprising phosphatidic acid.

90. The method of claim 88, wherein said composition comprises between about 7% and at least about 75% phosphatidic acid.

91. The method of claim 89, wherein said composition comprises between about 7% and at least about 75% phosphatidic acid.

92. The method according to claim 90, wherein said composition comprises between at least about 15% and at least about 50% phosphatidic acid.

93. The method according to claim 91, wherein said composition comprises between at least about 15% and at least about 50% phosphatidic acid.

94. The method according to claim 90, wherein said composition comprises between at least about 15% and at least about 30% phosphatidic acid.

95. The method according to claim 91, wherein said composition comprises between at least about 15% and at least about 30% phosphatidic acid.

96. The method according to claim 90, wherein said composition comprises between at least about 15% and at least about 25% phosphatidic acid.

97. The method according to claim 91, wherein said composition comprises between at least about 15% and at least about 25% phosphatidic acid.

98. The method according to claim 88 wherein the composition is in an administrable form.

99. The method according to claim 89 wherein the composition is in an administrable form.

100. The method according to claim 98, wherein said administrable form is selected from the group consisting of oral, topical, intraperitoneal, intraarticular, intracranial, intradermal, intramuscular, intraocular, intrathecal, intravenous, and subcutaneous.

101. The method according to claim 99, wherein said administrable form is selected from the group consisting of oral, topical, intraperitoneal, intraarticular, intracranial, intradermal, intramuscular, intraocular, intrathecal, intravenous, and subcutaneous.

102. The method according to claim 100 adapted for oral administration.

103. The method according to claim 101 adapted for oral administration.

104. The method according to claim 88, wherein said composition further comprises at least one antioxidant.

105. The method according to claim 89, wherein said composition further comprises at least one antioxidant.

106. The method according to claim 88, wherein said composition further comprises PS.

107. The method according to claim 89, wherein said composition further comprises PS.

108. A method for achieving abstinence from smoking cigarettes or other forms of tobacco for a period of at least 28 consecutive days, said method comprising administering to an individual smoker an effective amount of a composition comprising phosphatidic acid, and a pharmaceutically acceptable diluent, binder, filler, disintegrate, flavoring agent, coloring agent, lubricant or preservative.

109. A method for smoking cessation or promoting smoking cessation, said method comprises administering to an individual smoker an effective amount of a composition comprising at least about 16% (w/w) phosphatidic acid, at least one antioxidant and a pharmaceutically acceptable diluent, binder, filler, disintegrate, flavoring agent, coloring agent, lubricant or preservative.

110. The method according to claim 103 wherein said composition further comprises PS.

111. The method according to claim 104 wherein said composition further comprises PS.

112. A kit for smoking cessation, said kit comprises an administrable form of a composition comprising between at least about 10% and 22% (w/w) phosphatidic acid and instructions for use.

113. A kit for smoking cessation, said kit comprises an administrable form of a composition comprising at least about 22% (w/w) phosphatidic acid and instructions for use.

114. The kit according to claim 107, wherein said administrable form is selected from the group consisting of oral, topical, intraperitoneal, intraarticular, intracranial, intradermal, intramuscular, intraocular, intrathecal, intravenous, and subcutaneous.
